(19) United States
(12) Patent Application Publication
(10) Pub. No.: US 2011/0137063 A1
(43) Pub. Date: Jun. 9, 2011

(54) COMPOUNDS AND PROCESSES FOR PRODUCTION OF RADIOPHARMACEUTICALS

(75) Inventors: Ananth Srinivasan, Berlin (DE); Mathias Berndt, Berlin (DE); Keith Graham, Berlin (DE); Matthias Friebe, Berlin (DE); Heribert Schmitt-Willich, Berlin (DE)

(73) Assignee: BAYER SCHERING PHARMA AKTIENGESELLSCHAFT, BERLIN (DE)

(21) Appl. No.: 13/002,123

(22) PCT Filed: Jun. 24, 2009
(86) PCT No.: PCT/EP09/04537

§ 371 Date: Feb. 22, 2011

(30) Foreign Application Priority Data
Jul. 3, 2008 (EP) 08159601.7

(51) Int. Cl. C07C 309/77; C07C 309/80; C07C 309/86; C07C 309/68; C07C 309/76; C07C 209/74; C07C 227/18; C07C 209/84; C07C 227/40

(52) U.S. Cl. 558/49; 562/834; 558/51; 558/48; 564/412; 562/571; 562/554

(57) ABSTRACT

The present invention relates to novel perfluorinated precursors for the production of F-18 labeled radiotracers for Positron Emission Tomography (PET) and processes for radiolabeling and purification using these precursors. The invention also comprises radiopharmaceutical kits using these precursors and processes.

### FIELD OF THE INVENTION

The present invention relates to novel perfluorinated precursors for the production of F-18 labeled radiotracers for Positron Emission Tomography (PET) and processes for radiolabeling and purification using these precursors. The invention also comprises radiopharmaceutical kits using these precursors and processes.

### BACKGROUND OF THE INVENTION

Due to its favorable half-life of 110 minutes and the low β⁺ energy (635 keV) F-18 is currently the most important isotope for Positron Emission Tomography (Wüst, F. (2005) Amino Acids, 29, 323-339.) However, the relatively short half-live requires fast processes for synthesis and purification of F-18 labeled compounds.

Nucleophilic substitution reaction using [F-18]fluoride sources usually employ non-radioactive organic precursors (R-L) in amounts that are in large excess relative to the amount of the radiolabeling agent used. Excess precursors (R-L) must then be removed from the reaction mixture before the radiolabeled compound (R-¹⁸F) can be applied to a patient for diagnostic applications, because R-L can compete with and therefore interfere with binding of R-¹⁸F to its target. If this competition occurs, this effect may lead to suboptimal performance characteristics of the radiopharmaceutical. This is particularly the case for receptor-binding (i.e. specific targeting) radiopharmaceuticals.

$$R \text{-} L {}^{[ F \text{-} 18 ] fluoride source} R \text{-} {}^{18} F$$

R=targeting substrate
L=leaving group

The purification of R-L from R-¹⁸F is commonly accomplished by employing a chromatographic, e.g., HPLC, purification procedure. However, this technique requires specialized equipment and can moreover be tedious and time-consuming. Considering the half-life of most clinically useful radioisotopes, it is desirable to complete the radiosynthesis and purification prior to administration to a patient as rapidly as possible.

For robust and reliable routine production of F-18 labeled radiopharmaceuticals is a need in the art for purification techniques which offer rapid and efficient separation of unwanted species from the final pharmaceutical R-¹⁸F.

Solid phase processes for the production of ¹⁸F-radiolabelled tracers suitable for use as positron emission tomography radiotracers are for example disclosed in WO 2003/002157.

Although solid phase-supported nucleophilic substitution technologies can simplify purification steps substantially, they suffer from the inherent drawback that heterogeneous reaction conditions are usually less efficient, leading to poor radiochemical yields and slower reaction times compared to reactions carried out in solution, i.e., without a solid support. Hence some radiolabeling approaches with efficient purification strategies have been devised for substitution reactions conducted under homogenous conditions.

WO 2005/107819 relates to the purification of a radiolabelled tracer vector-X-R* resulting from a substitution reaction of R* for Y on the substrate vector-X-Y, using a solid support-bound scavenger group (scavenger resin). The scavenger resin Z-resin undergoes a similar substitution reaction on the non-reacted substrate vector-X-Y to displace Y and generate vector-X-Z-resin, which can be filtered off from the product vector-X-R* (which remains in solution). Hence, the purification procedure separates product from unreacted precursor. Alas, the scavenger resins are only designed to displace the moiety Y of the reactive group. In other words, this approach is limited to remove non-reacted precursors but does not allow a simultaneous removal of Y leaving group from the product. Furthermore, the reactive moiety Z of the scavenger resin described in WO 2005/107819 is limited only to groups that are good substitution agents for Y.

U.S. Ser. No. 61/044,550 describes purification strategies for nucleophilic substitution reactions in which the leaving group has an appended purification moiety, which allows for efficient separation of species containing the purification moiety (e.g. unreacted precursor) from species not containing the purification moiety (e.g. substituted product).

In organic chemistry, perfluorinated moieties are used on reagents and catalyst to allow easy purification processes. Combinatorial chemistry for the synthesis of compound libraries makes use of perfluoro tags of different length. Different applications are summarized in J. A. Gladysz, D. P. Curran, I. T. Horvath (eds), Handbook of fluorous chemistry. Wiley-VHC, Weinheim.

Donavan et al. ((J. Am. Chem. Soc., 2006, 128, 3536-3537)) describe a “homogeneous” soluble supported procedure for electrophilic radioiodine substitution utilizing a fluorine-rich soluble support wherein a leaving group is linked to a perfluorinated moiety. The radioiodinated product was isolated from both unreacted substrate and the leaving group based on the strong affinity of the perfluorinated moiety for other perfluorinated species. Although this homogeneous substitution procedure with fluorous-based purification has been demonstrated effective for electrophilic radioiodination, it is generally deemed not useful for nucleophilic [F-18]fluorinations because of the possibility of F-18/F-19 exchange. Exchange of F-19 for F-18 is well-known during F-18 radiolabeling reactions leading to low specific activity and poorer radiochemical yield of the product R-¹⁸F. F-18/F-19 exchange was found to be a particularly difficult problem when trying to incorporate perfluorinated materials (such as Teflon) into a F-18 radiotracer manufacturing process (e.g. J. Römer et al. Appl. rad. Isot. 55 (2001) 631-639.)

Thus, the technical problem to be solved by the invention is to provide a homogeneous soluble supported procedure for nucleophilic radiofluorination wherein the leaving groups as well as remaining educts can be easily and quickly separated from the product. Surprisingly, we found, despite the teaching of the prior art, that it is possible to use perfluorinated leaving groups for the synthesis of F-18 labeled radiopharmaceuticals to obtain radiotraces with radiochemical yields and specific activities acceptable for their use as imaging agents for Positron Emission Tomogaphy.

### DESCRIPTION OF THE INVENTION

In one aspect, the present invention relates to compounds of formula I

R-L-M (I)

wherein
R is a targeting substrate,
L is a leaving group suitable for a substitution with [F-18]fluoride,
M is a perfluorinated substituent, bearing 6-30 fluorine atoms,
One or more than one M groups can be attached to L.

In certain aspects of the present invention, R can be but is not limited to the group consisting of
a synthetic pharmaceutically active compound (drug), a metabolite, a signaling molecule, a hormone, a peptide, a protein, a receptor antagonist, a receptor agonist, a receptor inverse agonist, a vitamin, an essential nutrient, an amino acid, a fatty acid, a lipid, a nucleic acid, a mono-, di-, tri- or polysaccharide, and a steroid.

R-L-M is not
1,1,2,2,3,3,4,4,5,5,6,6,7,7,8,8,8-Heptadecafluoro-octane-1-sulfonic acid 2-(2-sulfamoyl-benzothiazol-6-yloxy)-ethyl ester, 1,1,2,2,3,3,4,4,5,5,6,6,7,7,8,8,8-Heptadecafluoro-octane-1-sulfonic acid 2-(4-sulfamoyl-phenyl)-ethyl ester, 1,1,2,2,3,3,4,4,5,5,6,6,7,7,8,8,8-Heptadecafluoro-octane-1-sulfonic acid 4-sulfamoyl-benzyl ester, Perfluoro-alkyl-1-sulfonic acid 1,2-bis-(aryl)-2-(perfluoroalkyl-1-sulfonyloxy)-vinyl ester, hexahydro-2,6-bis(perfluoroalkylsulfonyloxy)-cyclopenta[1,2-c:4,5-c′]dipyrrole-1,3,5,7(2H,6H)-tetrone, 3a,4,4a,7,8,8a-hexahydro-2,6-bis(perfluoroalkylsulfonyloxy)-4,8-ethenobenzol[1,2-c:4,5-c′]dipyrrole-1,3,5,7(2H,6H)-tetrone.
In preferred embodiments, R specifically binds to
   a receptor or enzyme or integrin or is specifically transported by a transporter that is preferentially expressed at a pathologic site within the mammalian body, preferably wherein the receptor or enzyme or integrin or transporter is exclusively expressed at a pathologic site within the mammalian body.
In preferred embodiments, R binds specifically to a site of
   infection, inflammation, cancer, platelet aggregation, angiogenesis, necrosis, ischemia, or tissue hypoxia, angiogenic vessels, Alzheimer's disease plaques, atherosclerotic plaques, pancreatic islet cells, thrombi, serotonin transporters, neuroepinephrin transporters, LAT1 transporters, apoptotic cells, macrophages, neutrophils, EDB fibronectin, receptor tyrosine kinases, or cardiac sympathetic neurons.
In particular embodiments of the present invention, examples of R include but are not limited to
   pharmaceutically active compounds (drugs), peptides, metabolites, signaling molecules, proteins, receptor antagonists, receptor agonists, receptor inverse agonists, vitamins, essential nutrients, amino acids, fatty acids, lipids, nucleic acids, steroids, hormones, glucose, galactose, fructose, mannitol, sucrose, stachyose, sorbose, and derivatives thereof, glutamine, glutamate, tyrosine, leucine, methionine, tryptophan, acetate, choline, thymidine, folate, methotrexate, Arg-Gly-Asp peptides, chemotactic peptides, alpha melanotropin peptide, somatostatin, bombesin, human pro-insulin connecting peptides and analogues thereof, GPIIb/IIIa-binding compounds, PF4-binding compounds, αvβ3, αvβ 6, or α4β1 integrin-binding compounds, somatostatin receptor binding compounds, GLP-1 receptor binding compounds, sigma 2 receptor binding compounds, sigma 1 receptor binding compounds, peripheral benzodiazepine receptor binding compounds, epidermal growth factor receptor binding compounds, PSMA binding compounds, estrogen receptor binding compounds, androgen receptor binding compounds, serotonin transporter binding compounds, neuroepinephrine transporter binding compounds, dopamine transporter binding compounds, LAT1 transporter binding compounds, and any combinations thereof.
As would be apparent to persons skilled in the art, it might be necessary to protect functional groups of R-L-M during the radiolabeling process. Such protection groups my be introduced by standard protection group chemistry (T. W. Greene, P. G. M. Wuts, Protection Groups in Organic Chemistry, published be John Wiley & Sons Inc.).
In preferred embodiments, L-M is selected from the group comprising:
   a. O—SO₂-M attached to an alkylic carbon atom of R,
   b. ⁺I-aryl-M attached to an arylic carbon atom of R,
   c. ⁺NA₁A₂ attached to an arylic carbon atom of R.
   wherein A₁ and A₂ are selected from the group comprising:
   a. branched or non-branched (C₁-C₆)alkyl,
   b. M,
   c. branched or non-branched (C₁-C₆)alkyl-M.
In preferred embodiments, M is selected from the group comprising:
   a. T,
   b. aryl-T,
   c. O-T,
   d. branched or non-branched (C₁-C₆)alkyl-T,
   e. branched or non-branched (C₁-C₆)alkoxy-T,
   f. branched or non-branched (C₁-C₆)alkenyl-T,
   g. (CF₂)ₚ—O-T,
   h. (CF₂)ₚ—O-branched or non-branched (C₁-C₆)alkyl-T,
   i. (CF₂)ₚ-aryl-T,
   j. branched or non-branched (C₁-C₆)alkyl-aryl-T,
   k. aryl-branched or non-branched (C₁-C₆)alkyl-T,
   l. aryl-branched or non-branched (C₁-C₆)alkoxy-T,
   m. aryl-branched or non-branched (C₁-C₆)alkenyl-T.
   wherein p is 1-2 and
   T is selected from the group comprising:
      a. branched or non-branched (C₃-C₁₀)perfluoroalkyl,
      b. ((CF₂)ₜ—O)ₘ—(CF₂)ₚ—F,
      wherein t is 1-4 and m is 2-10.

The term “alkyl” refers to a linear or branched chain monovalent or divalent radical consisting of solely carbon and hydrogen, containing no unsaturation and having the specified number of carbons, such as methyl (C₁), ethyl (C₂), n-propyl (C₃), 1-methlyethyl ((C₃) iso-propyl), n-butyl (C₄), n-pentyl (C₅) and the like. “Alkenyl” and “alkynyl” are similarly defined, but contain at least one carbon-carbon double or triple bond, respectively.

The term “alkoxy” refers to a linear or branched chain monovalent or divalent radical consisting of solely carbon and hydrogen, containing no unsaturation, having the specified number of carbons, such as methyl (C₁), ethyl (C₂), n-propyl (C₃), 1-methlyethyl (C₃ iso-propyl), n-butyl (C₄), n-pentyl (C₅) and the like and an oxygen atom which acts as link to the corresponding moiety.

As used herein, the term “aryl”, by itself or as part of another group, refers to monocyclic or bicyclic aromatic or heteroaromatic groups containing from 6 to 12 carbons in the ring portion, preferably 6-10 carbons in the ring portion, such as phenyl, naphthyl or tetrahydronaphthyl or to groups having 5 to 14 ring atoms; 6, 10 or 14 Π electrons shared in a cyclic array; and containing carbon atoms and 1, 2, 3 or 4 oxygen, nitrogen or sulfur heteroatoms. Examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxythiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, carbolinyl, phenanthridinyl, acridinyl, perimidinyl, phenanthrolinyl, phenazinyl, isothiazolyl, phenothiazinyl, isoxazolyl, furazanyl and phenoxazinyl.

The aryl group may comprise of further substituents such as cyano, trifluoromethyl, chloro, fluoro, keto- or ester functionalities.

In more preferred embodiments, compounds of formula I are selected from the group comprising:

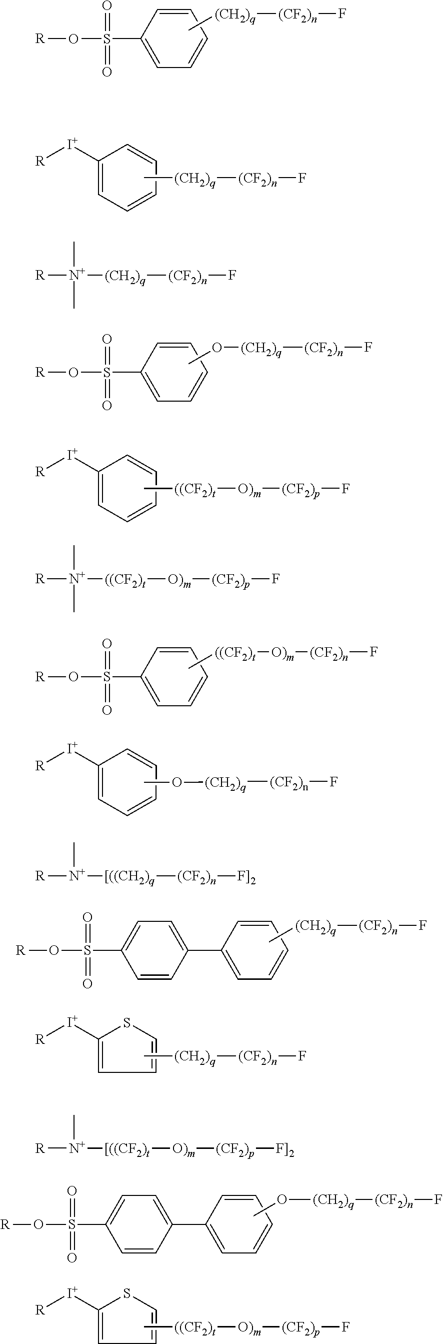

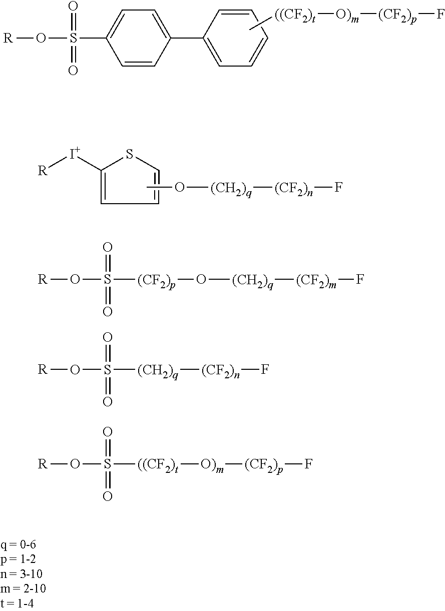

In a second aspect of the present invention compounds according to Formula II are provided for the use in a method for manufacturing a radiofluorinated compound. Furthermore, a method for manufacturing radiofluorinated compounds Q-¹⁸F by using a compound of formula II or a compound of formula I is provided.

Q-L-M (II)

wherein
Q is a organic moiety,
L is a leaving group suitable for a substitution with [F-18]fluoride,
M is a perfluorinated substituent, bearing 6-30 fluorine atoms,
One or more than one M groups can be attached to L.

L and M has the meaning as defined above.

In a preferred embodiment Q is selected from the group comprising:
a) R as defined above,
b) a moiety, allowing the use of Q-¹⁸F as agent for indirect fluorinations.

In a more preferred embodiment Q-¹⁸F is selected from the group comprising:
a) ¹⁸F—(C₁-C₆)alkyl-Y,
b) 4-[F-18]fluorobenzaldehyde,
c) 4-[F-18]fluorobenzoic acid, and esters thereof.

Wherein Y is selected from the group comprising:
a) halogen,
b) sulfonate (e.g. mesylate, tosylate, triflate, nonaflate).

In a preferred embodiment the present invention compounds according to Formula II are provided for the use in a method for manufacturing a diagnostic imaging agent by nucleophilic substitution.

The method of manufacturing can include a purification procedure as shown in Scheme 1.

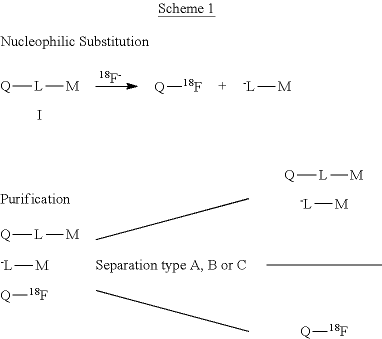

The method comprises the nucleophilic substitution step of reacting a compound of Formula II with [F-18]fluoride, and optionally thereafter the radiolabeled compound Q-¹⁸F can be converted into a pharmaceutically acceptable salt, hydrate or solvate thereof. Further, Q-¹⁸F is optionally further converted to Q′-¹⁸F. Because of the short half life of F-18 (110 minutes) the radiofluorinated compound must be prepared on the day of its clinical use. In the circumstances, the reactions steps are optimized for short time with yield as a secondary consideration. The reagents, solvents and conditions which can be used for this radiofluorination are common and well-known to the skilled person in the field (see e.g. J. Fluorine Chem. 27 (1985) 117-191). For example, F-18 labeling procedures using [F-18]fluoride and a base are well-established. Suitable bases are for example potassium carbonate or tetra alkyl ammonium carbonate. Complexing agents like Kryptofix™ (4,7,13,16,21,24-Hexaoxa-1,10-diazabicyclo[8.8.8]-hexacosane) or crown ethers could be used for the reaction. The preferred temperature is selected from the range RT to 180° C. It is known to someone skilled in the art that the radiofluorination reaction can be carried out, for example in a typical reaction vessel (e.g. Wheaton vial) or in a microreactor. The reaction can be heated by typical methods, e.g. oil bath, heating block or microwave. It is possible to carry out the reaction manually in a so-called hot cell and/or in an automated way using module synthesis (review: Krasikowa, Synthesis Modules and Automation in F-18 labeling (2006), in: Schubiger P. A., Friebe M., Lehmann L., (eds), PET-Chemistry—The Driving Force in Molecular Imaging. Springer, Berlin Heidelberg, pp. 289-316).

A second step of the method comprises a purification utilizing the perfluorinated moiety M that allows an easy separation of species that contain M from species that do not contain M. While not limited to these embodiments, the present invention is illustrated by describing a variety of separation types in more detail.

Purification Type a: Solid Phase Purification.
Species containing M can be separated from species not containing M by solid phase extraction using a resin or solid S functionalized with perfluorinated moieties or cartridges or columns made of such material. In this type of purification, M-containing species will bind preferentially to S.
In a preferred embodiment, S comprises perfluorinated reversed phase silica gel.

Purification Type B: Liquid Phase Extraction.
Species containing M can be separated from species not containing M by liquid phase extraction using a perfluorinated solvent. In this type of purification, M-containing species will partition preferentially into the perfluorinated solvent phase.

Purification Type C: Distillation.
Species containing M can be separated from species not containing M by distillation of species not containing M from the reaction mixture or distillation of species containing M from the reaction mixture.

Optionally, after radiolabeling, protecting groups may be removed by procedures which are standard in the art (T. W. Greene, P. G. M. Wuts, Protection Groups in Organic Chemistry, published be John Wiley & Sons Inc.)

Removal of protecting groups could be prior to or after the separation/purification (of species not containing M from species containing M) step.

A third aspect the present invention provides a radiopharmaceutical kit comprising compound of formula I.

The invention relates to a process of preparing a fluorine-18 labeled compound Q-¹⁸F comprising nucleophilic fluorination of compound of formula II Q-L-M, wherein
Q is an organic moiety,
L is a leaving group suitable for a substitution with [F-18]fluoride,
M is a perfluorinated substituent, bearing 6-30 fluorine atoms.

This process involves in a preferred embodiment a purification step. In a further embodiment this purification step removes remaining educts as well as leaving groups.

In a further embodiment the purification step makes use of the properties of perfluorinated moiety M.

In an even more preferred embodiment perfluorinated solid or liquid phase is used for the purification step.

The invention relates to process of preparing a fluorine-18 labeled compound R-¹⁸F comprising fluorination of compound of formula II with a [F-18]fluoride ion source.

In a preferred embodiment in this process the fluoride ion source may be selected from the group comprising:
a. potassium fluoride
b. caesium fluoride
c. tetraalkylammonium fluoride

In a further embodiment, Kryptofix™ is used in the fluorination process.

In a further embodiment, the fluorination reaction is carried out in homogeneous solution.

If desirable, the radiolabeled compound Q-¹⁸F may further be converted to the final product Q′-¹⁸F.

In a preferred embodiment, the process comprises a purification step.

In a further preferred embodiment, the purification step is a solid phase extraction.

In an even more preferred embodiment, the purification step is a solid phase extraction using a perfluorinated stationary phase.

The purification step may also be a liquid phase extraction.

In an even ore preferred embodiment, the purification step is a liquid phase extraction using a perfluorinated solvent.

The invention also relates to a method of manufacturing a radiolabeled compound, wherein the radiolabeled compound Q-¹⁸F is further converted to the final product Q′-¹⁸F after purification of Q-¹⁸F.

Furthermore, another aspect of the present invention relates to kits for carrying out a nucleophilic substitution and/or purification according to the present invention. In one embodiment, a kit according to the invention comprises at least a purification moiety M or a moiety L-M to be attached to Q-L or Q, respectively. In another embodiment, a kit comprises at least Q-L and a purification moiety M. In yet another embodiment, a kit according to the present invention comprises at least a moiety Q-L-M. Optionally, kits according to the present invention comprise a product manual, one or more compounds or resins to carry out a purification step and/or suitable reaction or purification media and the like.

In particular, the invention comprises:
1. A compound of Formula I:

R-L-M(ₓ) (I)
wherein
R is a targeting substrate.
L is a leaving group suitable for a substitution with [F-18]fluoride,
M is a perfluorinated substituent, bearing 6-30 fluorine atoms,
wherein X has the value of at least 1, more preferably X may have the value of 1, 2, 3, or 4,
and wherein R-L-M is not
   1,1,2,2,3,3,4,4,5,5,6,6,7,7,8,8,8-Heptadecafluoro-octane-1-sulfonic acid 2-(2-sulfamoyl-benzothiazol-6-yloxy)-ethyl ester, 1,1,2,2,3,3,4,4,5,5,6,6,7,7,8,8,8-Heptadecafluoro-octane-1-sulfonic acid 2-(4-sulfamoyl-phenyl)-ethyl ester, 1,1,2,2,3,3,4,4,5,5,6,6,7,7,8,8,8-Heptadecafluoro-octane-1-sulfonic acid 4-sulfamoyl-benzyl ester, Perfluoro-alkyl-1-sulfonic acid 1,2-bis-(aryl)-2-(perfluoroalkyl-1-sulfonyloxy)-vinyl ester, hexahydro-2,6-bis(perfluoroalkylsulfonyloxy)-cyclopenta[1,2-c:4,5-c′]dipyrrole-1,3,5,7(2H,6H)-tetrone, 3a,4,4a,7,8,8a-hexahydro-2,6-bis(perfluoroalkylsulfonyloxy)-4,8-ethenobenzol[1,2-c:4,5-c′]dipyrrole-1,3,5,7(2H,6H)-tetrone.
2. A compound of Formula I, wherein more than one M are attached to L.
3. A compound according to count 1-2, wherein R is a targeting moiety that specifically binds to a target site in a mammalian body.
4. A compound according to counts 1-3, wherein R is selected from the group consisting of a synthetic small molecule, a pharmaceutically active compound (drug), a metabolite, a signaling molecule, an hormone, a peptide, a protein, a receptor antagonist, a receptor agonist, a receptor inverse agonist, a vitamin, an essential nutrient, an amino acid, a fatty acid, a lipid, a nucleic acid, a mono-, di-, tri- or polysaccharide, and a steroid.

5. A compound according to count 1-4, wherein R specifically binds to a receptor or enzyme or integrin or is specifically transported by a transporter that is preferentially expressed at a pathologic site within the mammalian body, preferably wherein the receptor or enzyme or integrin or transporter is exclusively expressed at a pathologic site within the mammalian body.

6. A compound according to count 1-5, wherein R binds specifically to a site of infection, inflammation, cancer, platelet aggregation, angiogenesis, necrosis, ischemia, or tissue hypoxia, angiogenic vessels, Alzheimer's disease plaques, atherosclerotic plaques, pancreatic islet cells, thrombi, serotonin transporters, neuroepinephrin transporters, LAT1 transporters, apoptotic cells, macrophages, neutrophils, EDB fibronectin, receptor tyrosine kinases, or cardiac sympathetic neurons.
7. A compound according to count 1-6, wherein R is selected from the group consisting of synthetic small molecules, pharmaceutically active compounds (drugs), peptides, metabolites, signaling molecules, proteins, receptor antagonists, receptor agonists, receptor inverse agonists, vitamins, essential nutrients, amino acids, fatty acids, lipids, nucleic acids, steroids, hormones, glucose, galactose, fructose, mannitol, sucrose, stachyose, sorbose, and derivatives thereof, glutamine, glutamate, tyrosine, leucine, methionine, tryptophan, acetate, choline, thymidine, folate, methotrexate, Arg-Gly-Asp peptides, chemotactic peptides, alpha melanotropin peptide, somatostatin, bombesin, human pro-insulin connecting peptides and analogues thereof, GPIIb/IIIa-binding compounds, PF4-binding compounds, αvβ3, αvβ6, or α4β1 integrin-binding compounds, somatostatin receptor binding compounds, GLP-1 receptor binding compounds, sigma 2 receptor binding compounds, sigma 1 receptor binding compounds, peripheral benzodiazepine receptor binding compounds, epidermal growth factor receptor binding compounds, PSMA binding compounds, estrogen receptor binding compounds, androgen receptor binding compounds, serotonin transporter binding compounds, neuroepinephrine transporter binding compounds, dopamine transporter binding compounds, LAT1 transporter binding compounds, and any combinations thereof.
8. A compound according to counts 1-7, wherein L is O—SO₂ and L is attached to an alkylic carbon atom of R.
9. A compound according to counts 1-7, wherein L is I⁺Ar and L is attached to an aromatic carbon atom of R,
   wherein:
   Ar is an aromatic moiety or a heteroaromatic moiety
10. A compound according to count 9, wherein Ar is selected from the group comprising:
   a. (substituted) phenyl,
   b. (substituted) thiophene.
11. A compound according to counts 1-7, wherein L is N⁺A₁A₂ and L is attached to an aromatic carbon atom of R.
   wherein A₁ and A₂ are selected from the group comprising:
      a. branched or non-branched (C₁-C₆)alkyl,
      b. M,
      c. branched or non-branched (C₁-C₆)alkyl-M.
12. A compound according to count 11, wherein A₁ is methyl and A₂ is M
13. A compound according to counts 1-11, wherein M is selected from the group comprising:
   a. T
   b. Ar-T
   c. O-T
   d. branched or non-branched (C₁-C₆)alkyl-T
   e. branched or non-branched (C₁-C₆)alkoxy-T
   f. branched or non-branched (C₁-C₆)alkenyl-T
   g. (CF₂)ₚ—O-T
   h. (CF₂)ₚ—Ar-T
   i. branched or non-branched (C₁-C₆)alkyl-Ar-T
wherein:
n is 1-2
T is selected from the group comprising:
   a. branched or non-branched (C₃-C₁₀)perfluoroalkyl
   b. t(CF₂)ₜ—O)ₘ—(CF₂)ₚ—F
t is 1-4
m=3-10
p is 1-2
14. A process of preparing a fluorine-18 labeled compound R-¹⁸F comprising fluorination of compound of formula I according to counts 0-0 with a [F-18]fluoride ion source.
15. A process according to count 14 wherein the fluoride ion source is selected from the group comprising:
   d. potassium fluoride
   e. caesium fluoride
   f. tetraalkylammonium fluoride.
16. A process according to counts 14-15 wherein Kryptofix™ is used.
17. A process according to counts 14-16 wherein the fluorination reaction is carried out in homogeneous solution.
18. A process according to counts 14-17 wherein the radiolabeled compound R-¹⁸F is further converted to the final product R′-¹⁸F.
19. A process according to counts 14-18 comprising a purification step.
20. A process according to count 19 wherein the purification step is a solid phase extraction.
21. A process according to counts 19-20 wherein the purification step is a solid phase extraction using a perfluorinated stationary phase.
22. A process according to count 19 wherein the purification step is a liquid phase extraction.
23. A process according to counts 21 wherein the purification step is a liquid phase extraction using a perfluorinated solvent.
24. A process according to counts 14-23 wherein the radiolabeled compound R-¹⁸F is further converted to the final product R′-¹⁸F after purification of R-¹⁸F.
25. The kit for carrying out a process according to any one of counts 14 to 24 comprising a compound of formula I according to counts 1-13.
26. A kit for carrying out a process according to counts 14 to 24 comprising perfluorinated material for solid phase purification to separate M-containing species from a species that does not contain a M.
27. A kit for carrying out a process according to counts 14 to 24, further comprising a perfluorinated liquid extraction phase to separate M-containing species from a species that does not contain a M.
28. A process of preparing a fluorine-18 labeled compound Q-¹⁸F comprising nucleophilic fluorination of compound of formula II Q-L-M, wherein
   Q is an organic moiety,
   L is a leaving group suitable for a substitution with [F-18]fluoride,
   M is a perfluorinated substituent, bearing 6-30 fluorine atoms.
29. A process according to count 28 involving a purification step.
30. A process according to count 29 wherein the purification step makes use of the properties of perfluorinated moiety M.
31. A process according to count 30 wherein perfluorinated solid or liquid phase is used for the purification step.

### BRIEF DESCRIPTION OF THE DRAWINGS

FIGS. 1-5 are graphs of HPLC data.

### EXAMPLES

### Example 1

### Synthesis of 3-tridecafluorohexyl-benzenesulfonic acid 2-phenoxy-ethyl ester

Triethylamin (0.33 mL) and DMAP (20 mg) were added to a solution of 2-Phenoxy-ethanol (66 mg, 1 eg) and 3-Tridecafluorohexyl-benzenesulfonyl chloride (261 mg, 1.1 eg) in dichloromethane (5 mL). The reaction mixture was stirred for 16 h at room temperature. The solution was diluted with dichloromethane and carefully neutralized with 5% sodium bicarbonate solution. The phases were separated and the organic layer was washed with water and brine. The solution was dried over sodium sulfate and filtered. The crude product was purified by column chromatrography (silica gel, hexane/ethyl acetate=9.5/0.5) to afford 117 mg (41%) 3-tridecafluorohexyl-benzenesulfonic acid 2-phenoxy-ethyl ester as white solid.

### Example 2

### Radiofluorination of 3-tridecafluorohexyl-benzenesulfonic acid 2-phenoxy-ethyl ester, synthesis of 2-[F-18]fluoroethoxybenzene

[F-18]fluoride was trapped on a QMA cartridge. The activity was eluted using 1.5 mL kryptofix solution (5 mg kryptofix, 1 mg K₂CO₃, 1.25 mL acetonitrile, 0.25 mL water) into a 5 mL V-vial. The solution was dried at 120° C. under gentle nitrogen stream. Acetonitrile (2×1 mL) was added and the drying procedure was repeated. 2 mL acetonitrile was added to the residue and an aliquot of 0.5 ml was taken of and added to 2 mg 3-tridecafluorohexyl-benzenesulfonic acid 2-phenoxy-ethyl ester. The mixture was stirred for 15 min at room temperature.

### Crude Reaction Mixture:

(HPLC, Chromolith SpeedROD RP-18e, MeCN/Water, 0% MeCN-95% MeCN)

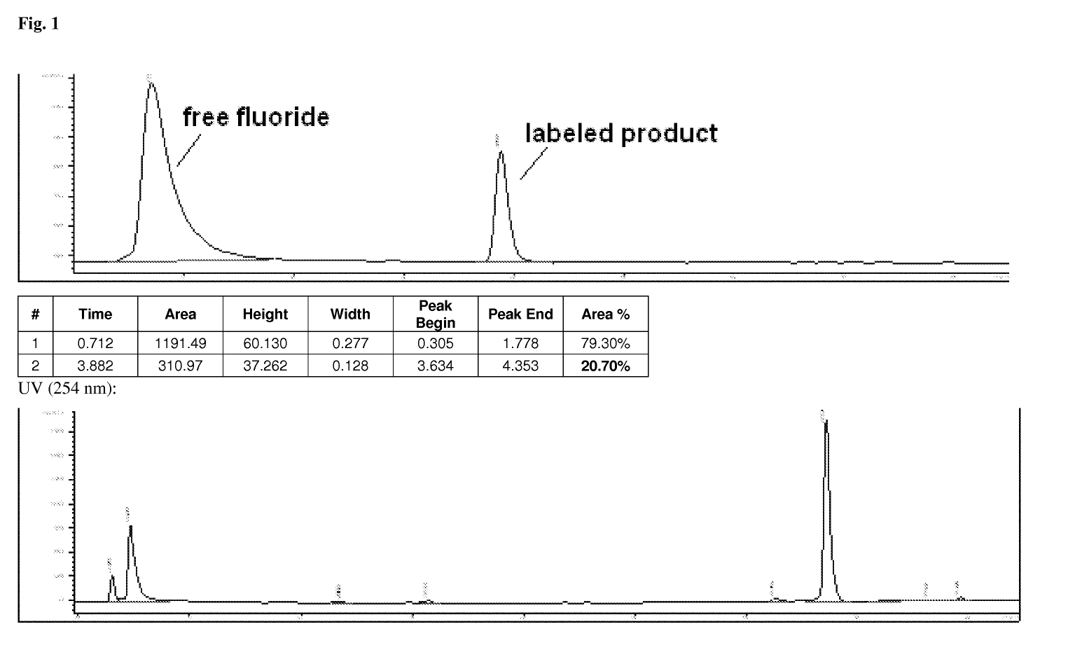

The activity is shown in FIG. 1.

The reaction mixture was diluted with water (10 mL) and passed through a perfluoro 8 and a C18 (tC18 plus, waters) cartridge. The cartridges were washed with water (5 mL) and the product was eluted with ethanol (2 mL).

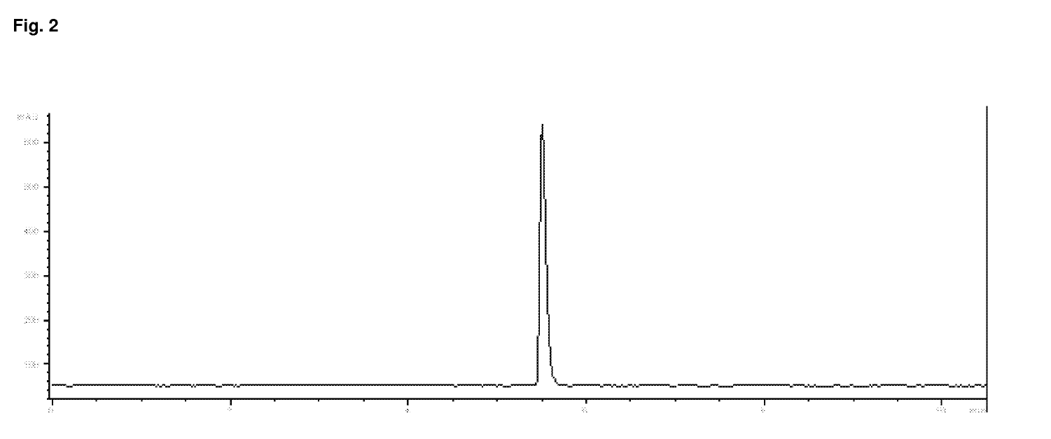

FIG. 2 shows HPLC, activity.

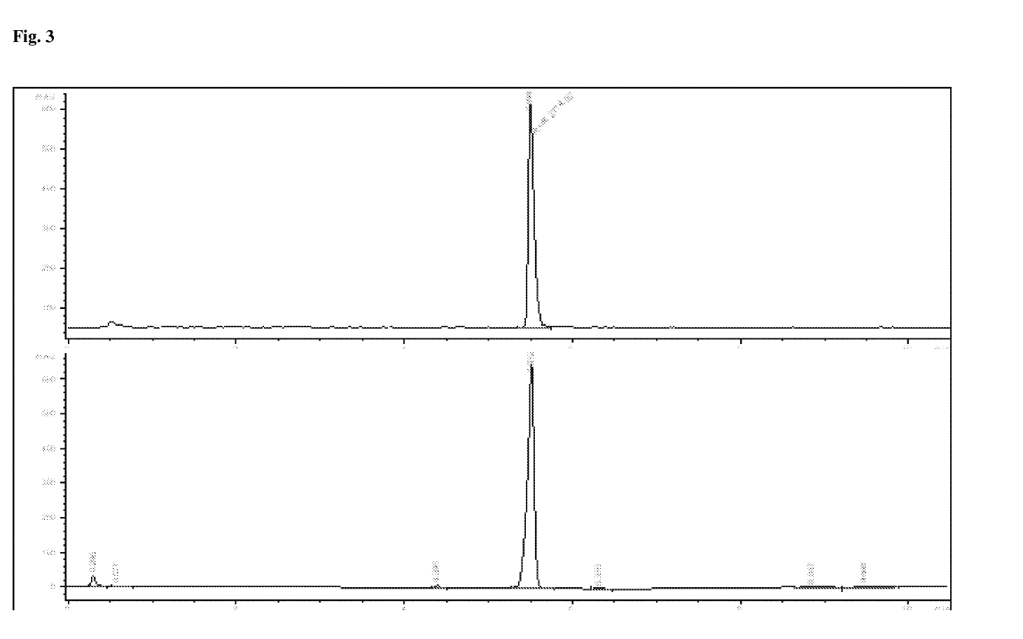

FIG. 3 shows HPLC, coelution with cold standard (F-19 compound), activity (top), UV (bottom).

### Example 3

### Synthesis of 1H,1H,2H,2H-perfluoroctyl-1-sulfonic acid chloride

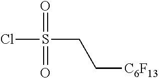

PCl₅ (486 mg, 2.34 mmol) was added to a suspension of 1H,1H,2H,2H-perfluoroctyl-1-sulfonic acid (33, 500 mg, 1.17 mmol) in 10 mL POCl₃. The mixture was stirred for 6 h at 60° C. and for 72 h at room temperature. The reaction mixture was diluted with dichloromethane and ice water and neutralized with saturated sodium bicarbonate solution. The organic layer was dried over sodium sulfate. After filtration the solvent was removed under reduced pressure. 1H,1H,2H,2H-perfluoroctyl-1-sulfonic acid chloride (477 mg) was obtained as yellow oil, that crystallized at room temperature. The crude product was used for subsequent reactions without further purification.

### Example 4

### Synthesis of 3-perfluorohexyl-benzenesulfonyl chloride

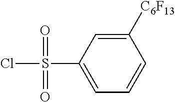

(Perfluorohexyl)benzene (18.7 g) was added drop wise to chlorosulfonic acid (22 mL) at 0° C. under nitrogen atmosphere. The mixture was stirred for 2.5 h at 0° C. Stirring was continued at room temperature for 18 h. The reaction mixture was carefully added to ice water (400 mL). It was extracted with diethyl ether (3×). The combined organic fractions were washed with 10% sodium bicarbonate solution and water, dried over magnesium sulfate, filtered and the solvent was evaporated. The crude product was purified by column chromatography (silica, hexane/ethyl acetate=97/3). 7.6 g 3-perfluorohexyl-benzenesulfonic acid were obtained as oil.

¹H-NMR (CDCl₃): δ=8.32-8.26 (2H), 7.99 (d, 1H), 7.84 (t, 1H).

3-perfluorohexyl-benzenesulfonic acid (1.05 g) was dissolved in 15.4 mL POCl₃. PCl₅ (528 mg) was added and the mixture was stirred at 60° C. for 6 h. The reaction mixture was diluted with dichloromethane and ice water was added. For neutralization, saturated sodium bicarbonate solution was added slowly. The mixture was extracted with dichloromethane (3×). The combined organic layers were dried over magnesium sulfate, filtrated and the solvent was evaporated. 863 mg 3-perfluorohexyl-benzenesulfonyl chloride were obtained as light yellow oil.

¹H-NMR (CDCl₃): δ=8.31-8.26 (2H), 7.98 (d, 1H), 7.84 (t, 1H).

### Example 5

### Synthesis of 3-perfluorooctyl-benzenesulfonyl chloride

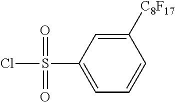

Iodobenzene (3.23 g) was dissolved in DMSO (32 mL) under N₂ atmosphere. Iodoperfluorooctane (4.2 mL) and copper powder (3.0 g) were added and the suspension was heated at 100° C. for 16 h. After cooling to room temperature, the mixture filtered (2× washed with diethyl ether) and the filtrate was hydrolyzed with 2M HCl. The mixture was extracted with diethyl ether and the combined organic fractions were washed with water, dried over sodium sulphate and the solvent was evaporated. The crude product was purified by column chromatography (silica, hexane) to afford 6.23 g (perfluorooctyl)benzene.

¹H-NMR (CDCl₃): δ=7.62-7.55 (3H), 7.54-7.47 (d, 2H).

(Perfluorooctyl)benzene (6.23 g) was added drop wise to chlorosulfonic acid (5.85 mL) at 0° C. under nitrogen atmosphere. The mixture was stirred for 2.5 h at 0° C. Stirring was continued at room temperature for 18 h. The reaction mixture was carefully added to ice water (400 mL). It was extracted with diethyl ether (3×). The combined organic fractions were washed with 10% sodium bicarbonate solution and water, dried over magnesium sulfate, filtered and the solvent was evaporated. The crude product was purified by column chromatography (silica, hexane/ethyl acetate=98/2). 2.0 g 3-perfluorooctyl-benzenesulfonic acid were obtained as light yellow solid.

¹H-NMR (CDCl₃): δ=8.31-8.26 (2H), 7.98 (d, 1H), 7.84 (t, 1H).

3-perfluorooctyl-benzenesulfonic acid (1.94 g) was dissolved in 23.5 mL POCl₃. PCl₅ (810 mg) was added and the mixture was stirred at 60° C. for 6 h. The reaction mixture was diluted with dichloromethane and ice water was added. For neutralization, saturated sodium bicarbonate solution was added slowly. The mixture was extracted with dichloromethane (3×). The combined organic layers were dried over magnesium sulfate, filtrated and the solvent was evaporated. 1.68 mg 3-perfluorooctyl-benzenesulfonyl chloride were obtained as light yellow oil.

¹H-NMR (CDCl₃): δ=8.31-8.26 (2H), 7.98 (d, 1H), 7.84 (t, 1H).

### Example 6

### Synthesis of 4′-heptadecafluorooctyl-biphenyl-4-sulfonsulfonic acid chloride

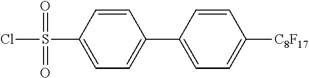

A suspension of 1-iodoperfluoroctane (2.73 g, 5.00 mmol), monoiodobiphenyl (1.40 g, 5.00 mmol) and copper powder (1.00 g, 15.6 mmol) in 10 mL DMSO was stirred for 12 h at 100° C. under nitrogen atmosphere. After filtration the mixture was washed with 2M HCl and extracted with diethyl ether. The combined organic layers were washed with water and dried over sodium sulfate. The solvent was removed under reduced pressure. The crude mixture was purified by column chromatography (silica, hexane). 2.03 g 4-heptadecafluoroctyl-biphenyl were obtained as colorless powder

¹H-NMR (CDCl₃): δ=7.41 (t, J=7.3 Hz, 1H, Ar), 7.48 (t, J=7.3 Hz, 2H, Ar), 7.59-7.64 (m, 2H, Ar), 7.66 (d, J=8.6 Hz, 2H, Ar), 7.72 (d, J=8.6 Hz, 2H, Ar).

4-heptadecafluoroctyl-biphenyl (1.81 g, 3.17 mmol) was dissolved in 45 mL chloroform. Chlorosulfonic acid (235 μL, 3.53 mmol) was added drop wise. The mixture was stirred over night at room temperature. The solvent was evaporated and the crude product was re crystallized from acetonitrile. 1.06 g 4′-heptadecafluorooctyl-biphenyl-4-sulfonic acid were obtained as colourless solid.

To a suspension of 4′-heptadecafluorooctyl-biphenyl-4-sulfonic acid (1.00 g, 1.53 mmol) in 10 mL POCl₃ was added PCl₅ (364 mg, 1.75 mmol). The mixture was stirred for 6 h at 60° C. It was diluted with dichloromethane and ice water and neutralized with saturated sodium bicarbonate solution. The organic layer was dried over sodium sulfate and the solvent was evaporated. 820 mg 4′-heptadecafluorooctyl-biphenyl-4-sulfonsulfonic acid chloride were obtained as colorless solid and used in subsequent reactions without additional purification.

### Example 7

### Synthesis of 1H,1H,2H,2H-perfluoroctane-1-sulfonic acid 2-phenoxyethyl ester

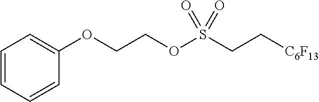

4-Dimethylaminopyridine (6.10 mg, 4.99 μmol), NEt₃ (219 μA 1.58 mmol) and 1H,1H,2H,2H-perfluoroctyl-1-sulfonic acid chloride (470 mg, 1.05 mmol) were added to a solution of 2-phenoxyethanol (132 μA 0.525 mmol) in 5 mL dichlormethane at 0° C.

The solution was stirred over night at room temperature. The mixture was diluted with dichloromethane and washed with saturated sodium chloride solution. The organic layer was dried over sodium sulfate. After filtration and evaporation of the solvent, the crude mixture was purified by column chromatography (silica, hexane(ethyl acetate=8/2). 365 mg 1H,1H,2H,2H-perfluoroctane-1-sulfonic acid 2-phenoxyethyl ester were obtained as colorless solid.

### Example 8

### Synthesis of 4′-heptadecafluorooctyl-biphenyl-4-sulfonic acid 2-phenoxy-ethylester

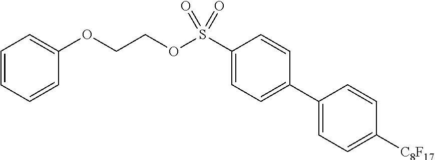

NEt₃ (93.0 μL, 670 μmol), 4-dimethylaminopyridine (2.60 mg, 21.3 μmol) and 4′-heptadecafluorooctyl-biphenyl-4-sulfonsulfonic acid chloride (300 mg) were added to a solution of 2-phenoxyethanol (28.2 μL, 225 μmol) in 5 ml dichloromethane at 0° C. The mixture was stirred for 6 h at 0° C. and 72 h at room temperature. After dilution with dichloromethane the solution was washed with saturated sodium chloride solution. The organic layer was dried over sodium sulfate and the solvent was evaporated. The crude product was purified by column chromatography (silica, hexane/ethyl acetate=9/1). 186 mg 4′-heptadecafluorooctyl-biphenyl-4-sulfonic acid 2-phenoxy-ethylester were obtained as colorless solid.

¹H-NMR (CDCl₃): δ=8.04 (d, 2H), 7.76 (d, 2H), 7.72 (4H), 7.26-7.22 (2H), 6.95 (t, 1 H), 6.77 (d, 2H), 4.48-4.46 (2H), 4.20-4.18 (2H).

### Example 9

### Synthesis of 3-perfluorooctyl-benzenesulfonic acid 2-{2-[2-(4-{(E)-2-[4-(tert-butoxycarbonyl-methyl-amino)-phenyl]-vinyl}-phenoxy)-ethoxy]-ethoxy}-ethyl ester

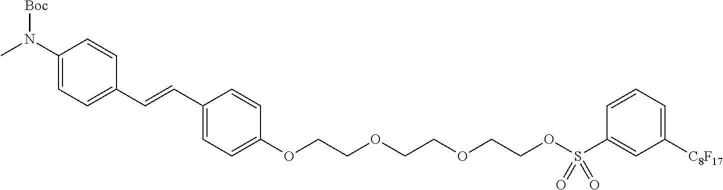

50 mg {4-[(E)-2-(4-{2-[2-(2-hydroxy-ethoxy)-ethoxy]-ethoxy}-phenyl)-vinyl]-phenyl}-methyl-carbamic acid tert-butyl ester were dissolved in 2 ml dichloromethane. DMAP (2.7 mg) and Et₃N (31 μl) were added and the mixture was cooled to 0° C. 3-perfluorooctyl-benzenesulfonyl chloride (97.5 mg) in 2 mL dichloromethane was added drop wise. The mixture was stirred over night at room temperature. The solvent was removed under reduced pressure and the crude product was purified by column chromatography (silica, hexane/ethyl acetate=8:2). 3-perfluorooctyl-benzenesulfonic acid 2-{2-[2-{4-1 (E)-2-[4-(tert-butoxycarbonyl-methyl-amino)-phenyl]-vinyl}-phenoxy)-ethoxy]-ethoxy}-ethyl ester (66 mg) was obtained as light yellow solid.

¹H-NMR (CDCl₃): δ=8.17-8.13 (2H), 7.86 (d, 1H), 7.72 (t, 1H), 7.45-7.41 (4H), 7.23-7.19 (2H), 7.04-6.87 (4H), 4.28-4.24 (2H), 4.15-4.11 (2H), 3.84-3.81 (2H), 3.74-3.71 (2H), 3.67-3.63 (2H), 3.62-3.59 (2H), 3.27 (3H), 1.46 (9H9.

### Example 10

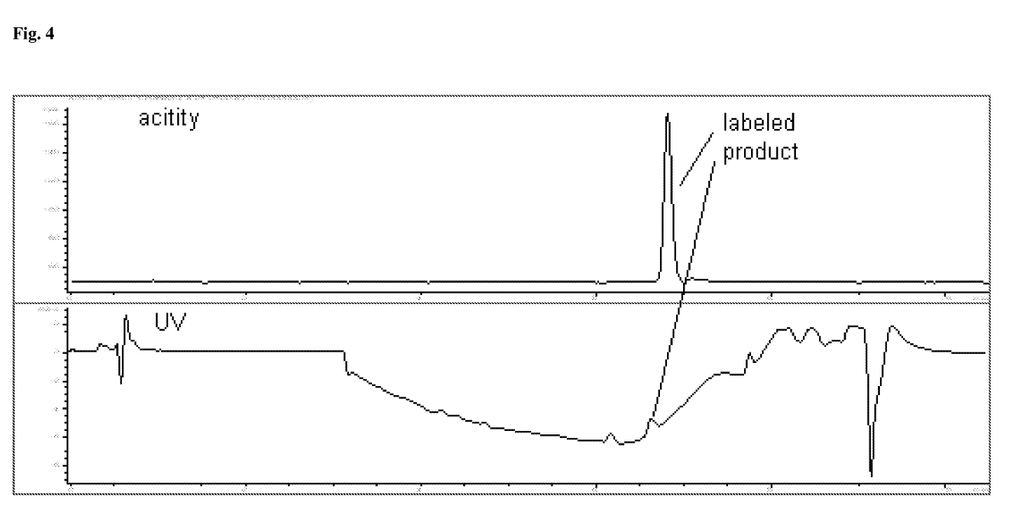

### Radiolabeling of 2-{2-[2-(4-{(E)-2-[4-(tert-butoxycarbonyl-methyl-amino)-phenyl]-vinyl}-phenoxy)-ethoxy]-ethoxy}-ethyl ester

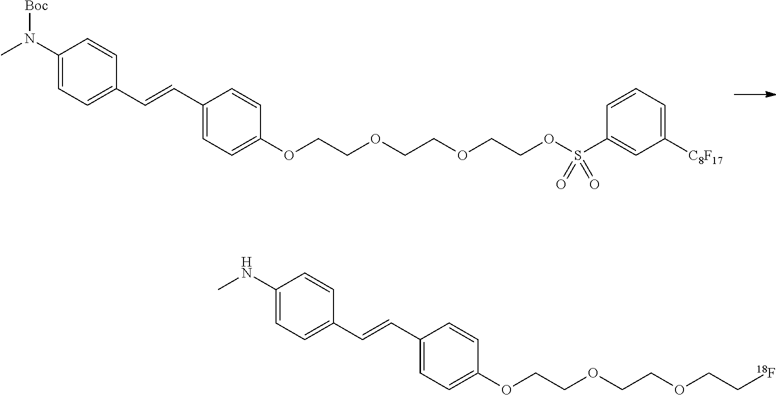

[F-18]Fluoride (1.3 GBq) was trapped on a QMA cartridge (SepPak light, waters). The activity was eluted with potassium carbonate/kryptofix solution (7.5 mg kryptofix, 1.5 mg potassium carbonate, 1425 μL acetonitrile, 75 μL water). The mixture was dried at 140° C. under gentle nitrogen stream. 2-{2-[2-(4-{(E)-2-[4-(tert-butoxycarbonyl-methyl-amino)-phenyl]-vinyl}-phenoxy)-ethoxy]-ethoxy}-ethyl ester (4 mg in 0.5 mL acetonitrile) was added and the resulting mixture was stirred at 30° C. for 15 min. Water was added and the mixture was passed through a perfluoro-C8 cartridge (Fluoroflash, Fluka) and a C18 cartridge (tC18 SepPak plus, waters). The cartridges were washed with water. The activity was eluted from the C18 cartridge with ethanole (312 MBq). HPLC analysis (C18) indicated complete separation for excess of precursor and only minor formation of side product through hydrolysis of the precursor. Results are shown in FIG. 4

### Example 11

### Synthesis of 2-tert-Butoxycarbonylamino-4-[3-(3-trifluoromethyl-benzenesulfonyloxy)-propyl]-pentanedioic acid di-tert-butyl ester

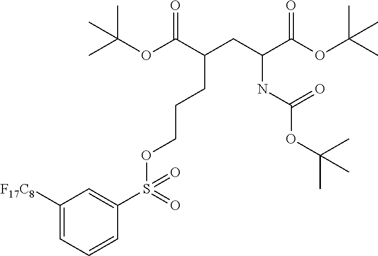

2-tert-Butoxycarbonylamino-4-(3-hydroxy-propyl)-pentanedioic acid di-tert-butyl ester (84 mg) was dissolved in dichloromethane (2 mL). NEt₃ (0.14 mL) and 3-perfluorooctyl-benzenesulfonyl chloride (119 mg) were added. The mixture was stirred at room temperature for 4 h. The crude product was purified by column chromatography (silica, hexane/ethyl acetate) to afford 120 mg 2-tert-Butoxycarbonylamino-4-[3-(3-trifluoromethyl-benzenesulfonyloxy)-propyl]-pentanedioic acid di-tert-butyl ester.

¹H-NMR (CDCl₃): δ=8.16-8.12 (2H), 7.92-7.87 (1H), 7.79-7.72 (1H), 4.89-4.82 (1 H), 4.14-4.05 (3H), 2.34-2.24 (1H), 1.92-1.54 (6H), 1.48-1.38 (27H).

### Example 12

### Radiolabeling of 2-tert-Butoxycarbonylamino-4-[3-(3-trifluoromethyl-benzenesulfonyloxy)-propyl]-pentanedioic acid di-tert-butyl ester

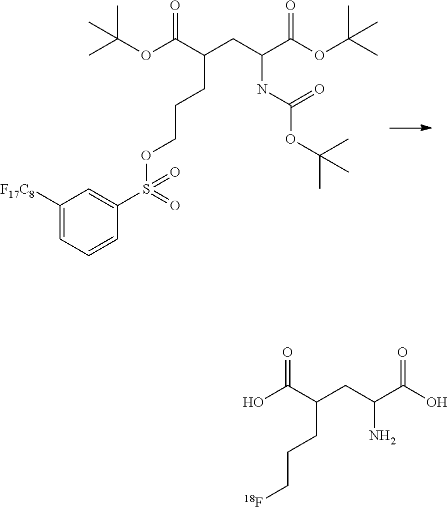

[F-18]Fluoride (459 MBq) was trapped on a QMA cartridge (SepPak light, waters). The activity was eluted with potassium carbonate/kryptofix solution (5 mg kryptofix, 1 mg potassium carbonate, 950 μL, acetonitrile, 50 μL water). The mixture was dried at 140° C. under gentle nitrogen stream. 2-tert-Butoxycarbonylamino-4-[3-(3-trifluoromethyl-benzenesulfonyloxy)-propyl]-pentanedioic acid di-tert-butyl ester (10 mg in 1 mL acetonitrile) was added and the resulting mixture was stirred at 40° C. for 15 min. Water was added and the mixture was passed through a perfluoro-C8 cartridge (Fluoroflash, Fluka) and a silica cartridge (SepPak plus, waters). The cartridges were washed with water/acetonitrile. The solution was concentrated at 110° C. under gentle nitrogen steam an hydrochloric acid was added. The mixture was heated for 10 min at 120° C. Solvent exchange was made by trapping the labelled product on a cation exchange cartridge (MCX, waters) and elution with isotonic phosphate buffer. The final product was analyzed by pre-column derivatization HPLC. No non-radioactive side products (derived from hydrolysis of the precursor) were obtained.

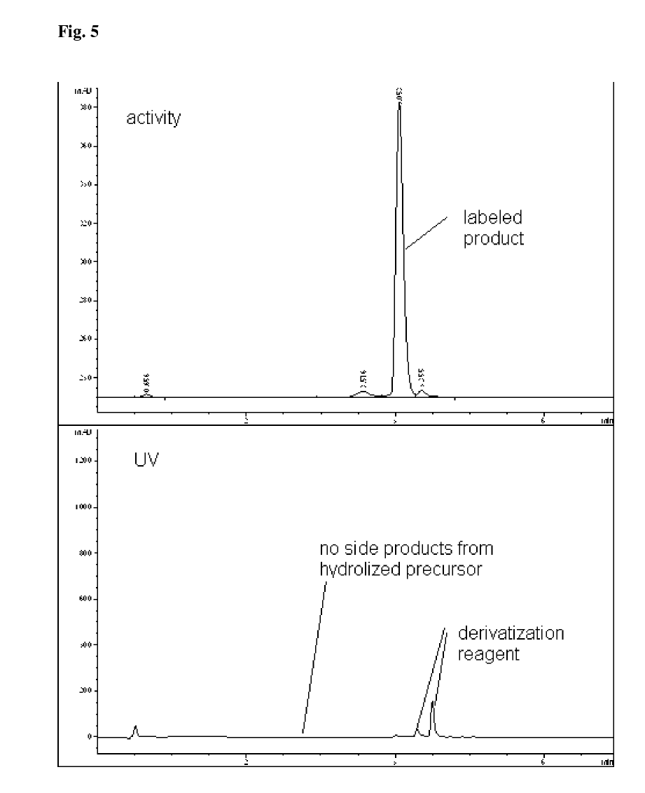

Results are shown in FIG. 5.

1. A compound of Formula I:
R-L-M(ₓ) (I)
wherein
R is a targeting substrate.
L is a leaving group suitable for a substitution with [F-18]fluoride,
M is a perfluorinated substituent, bearing 6-30 fluorine atoms,
X has the value of at least 1,
and wherein R-L-M is not
1,1,2,2,3,3,4,4,5,5,6,6,7,7,8,8,8-Heptadecafluoro-octane-1-sulfonic acid 2-(2-sulfamoyl-benzothiazol-6-yloxy)-ethyl ester, 1,1,2,2,3,3,4,4,5,5,6,6,7,7,8,8,8-Heptadecafluoro-octane-1-sulfonic acid 2-(4-sulfamoyl-phenyl)-ethyl ester, 1,1,2,2,3,3,4,4,5,5,6,6,7,7,8,8,8-Heptadecafluoro-octane-1-sulfonic acid 4-sulfamoyl-benzyl ester, Perfluoro-alkyl-1-sulfonic acid 1,2-bis-(aryl)-2-(perfluoroalkyl-1-sulfonyloxy)-vinyl ester, hexahydro-2,6-bis(perfluoroalkylsulfonyloxy)-cyclopenta[1,2-c:4,5-c′]dipyrrole-1,3,5,7(2H,6H)-tetrone, 3a,4,4a,7,8,8a-hexahydro-2,6-bis(perfluoroalkylsulfonyloxy)-4,8-ethenobenzol[1,2-c:4,5-c′]dipyrrole-1,3,5,7(2H,6H)-tetrone.

2. A compound according to claim 1, wherein R is selected from the group consisting of a synthetic small molecule, a pharmaceutically active compound (drug), a metabolite, a signaling molecule, an hormone, a peptide, a protein, a receptor antagonist, a receptor agonist, a receptor inverse agonist, a vitamin, an essential nutrient, an amino acid, a fatty acid, a lipid, a nucleic acid, a mono-, di-, tri- or polysaccharide, a steroid, hormones, glucose, galactose, fructose, mannitol, sucrose, stachyose, sorbose, and derivatives thereof, glutamine, glutamate, tyrosine, leucine, methionine, tryptophan, acetate, choline, thymidine, folate, methotrexate, Arg-Gly-Asp peptides, chemotactic peptides, alpha melanotropin peptide, somatostatin, bombesin, human pro-insulin connecting peptides and analogues thereof, GPIIb/IIIa-binding compounds, PF4-binding compounds, αvβ3, αvβ6, or α4β1 integrin-binding compounds, somatostatin receptor binding compounds, GLP-1 receptor binding compounds, sigma 2 receptor binding compounds, sigma 1 receptor binding compounds, peripheral benzodiazepine receptor binding compounds, epidermal growth factor receptor binding compounds, PSMA binding compounds, estrogen receptor binding compounds, androgen receptor binding compounds, serotonin transporter binding compounds, neuroepinephrine transporter binding compounds, dopamine transporter binding compounds, LAT1 transporter binding compounds, and any combinations thereof.

3. A compound according to claims 1-2, wherein R specifically binds to a receptor or enzyme or integrin or is specifically transported by a transporter that is preferentially expressed at a pathologic site within the mammalian body, preferably wherein the receptor or enzyme or integrin or transporter is exclusively expressed at a pathologic site within the mammalian body or wherein R specifically binds to a site of infection, inflammation, cancer, platelet aggregation, angiogenesis, necrosis, ischemia, or tissue hypoxia, angiogenic vessels, Alzheimer's disease plaques, atherosclerotic plaques, pancreatic islet cells, thrombi, serotonin transporters, neuroepinephrin transporters, LAT1 transporters, apoptotic cells, macrophages, neutrophils, EDB fibronectin, receptor tyrosine kinases, or cardiac sympathetic neurons.

4. A compound according to claims 1-3, wherein L is O—SO₂ and L is attached to an alkylic carbon atom of R.

5. A compound according to claims 1-4, wherein M is selected from the group comprising:
T
Ar-T
O-T
branched or non-branched (C₁-C₆)alkyl-T
branched or non-branched (C₁-C₆)alkoxy-T
branched or non-branched (C₁-C₆)alkenyl-T
(CF₂)ₚ—O-T
(CF₂)ₚ—Ar-T
branched or non-branched (C₁-C₆)alkyl-Ar-T
wherein:
n is 1-2
T is selected from the group comprising:
branched or non-branched (C₃-C₁₀)perfluoroalkyl
((CF₂)ₜ—O)ₘ—(CF₂)ₚ—F
t is 1-4
m=3-10
p is 1-2.

6. A process of preparing a fluorine-18 labeled compound R-¹⁸F comprising fluorination of compound of formula I according to claims 1-5 with a [F-18]fluoride ion source.

7. A process according to claim 6 wherein the fluoride ion source is selected from the group comprising:
potassium fluoride
caesium fluoride
tetraalkylammonium fluoride.

8. A process according to claims 6-7 wherein the fluorination reaction is carried out in homogeneous solution.

9. A process according to claims 6-8 comprising a purification step.

10. A process according to claim 9 wherein the purification step is a solid phase extraction.

11. A process according to claims 9-10 wherein the purification step is a solid phase extraction using a perfluorinated stationary phase.

12. A process according to claim 9 wherein the purification step is a liquid phase extraction.

13. A process according to claim 11 wherein the purification step is a liquid phase extraction using a perfluorinated solvent.

14. A process according to claims 6-13 wherein the radiolabeled compound R-¹⁸F is further converted to the final product R′-¹⁸F after purification of R-¹⁸F.

15. The kit for carrying out a process according to any one of claims 6 to 14 comprising a compound of formula I according to claims 1-5.

16. A process of preparing a fluorine-18 labeled compound Q-¹⁸F comprising nucleophilic fluorination of compound of formula II Q-L-M, wherein
Q is an organic moiety,
L is a leaving group suitable for a substitution with [F-18]fluoride,
M is a perfluorinated substituent, bearing 6-30 fluorine atoms,
Wherein the process involves a purification step, that makes use of the properties of perfluorinated moiety M.
